(19) United States
(12) Patent Application Publication
(10) Pub. No.: US 2024/0016707 A1
(43) Pub. Date: Jan. 18, 2024

(54) SOLUBLE SOLID FORM COSMETIC

(71) Applicant: CASTARALO LTD., Hong Kong (CN)

(72) Inventors: Laurie Anne Marie MIAS, Hong Kong (CN); Gaëtan MILIS, Spa (BE); Delphine ROBERTI, Spa (BE)

(73) Assignee: CASTARALO LTD., Hong Kong (CN)

(21) Appl. No.: 18/039,989

(22) PCT Filed: Dec. 10, 2021
(86) PCT No.: PCT/IB2021/061569

§ 371 Date: Jun. 2, 2023

(30) Foreign Application Priority Data
Dec. 10, 2020 (EP) 20212948.2

(51) Int. Cl. A61K 8/04; A61K 8/02; A61Q 19/00

(52) U.S. Cl. CPC A61K 8/04; A61K 8/0216; A61Q 19/00; A61K 2800/524; A61K 2800/80; A61K 2800/10; A61K 2800/57

(57) ABSTRACT

The invention relates to a composition in solid form comprising cosmetic actives which is soluble and a method for producing the same. Also provided is a kit comprising two or more compositions in solid form and a vial for reconstituting, holding and dispensing a fluid cosmetic.

### FIELD

The present invention relates to a new formulation of fluid cosmetics which allows for a reduction in packaging and waste.

### BACKGROUND

The cosmetic industry provides cosmetic products in ready-to-use formats and formulations. In general, cosmetic products for the skin, body and hair are in the form of aqueous dispersions or emulsions provided in relatively large packaging units. The size of the packaging has several disadvantages in that it requires a high amount of packaging material and takes up space in storage and transport. Large amounts of packaging material and space has negative effect on the environment. In addition, the vials containing the cosmetics are for single use which, even if to some extent recyclable, also generates a lot of waste.

Accordingly, there is a need for cosmetic products which have a reduced impact on the environment.

### SUMMARY

The present invention is in particular captured by the below independent embodiments.

In a first aspect the application provides compositions in solid form comprising a cosmetic agent, for dilution into a fluid cosmetic agent, such as water. More particularly, the composition comprises one or more water-soluble and dispersible cosmetic active agent and additional ingredients which ensure that it can be formulated into a solid form into a stable fluid cosmetic agent. In particular embodiments, the composition comprises one or more water-soluble and dispersible cosmetic active agents and one or more agglomerating agents or carriers, one or more water-soluble preservatives, one or more water-soluble pH-modifying agents, and optionally one or more water-soluble surfactants. In particular embodiments, the composition in solid form comprises 20-90% carrier such as saccharides, more particularly maltodextrine and/or dissacharides, 1-10% water-soluble preservative, 1-10% of one or more water-soluble and dispersible cosmetic active agent and optionally 0.1-10% of one or more water-soluble pH-modifying agents and 1-20% of one or more water-soluble surfactants.

In particular embodiments, the cosmetic active agent is a skincare product. In further particular embodiments, the amount of the water-soluble preservative, water-soluble pH modifying agent, water-soluble surfactant (if present) and said water-soluble and dispersible cosmetic actives in said solid form corresponds to the amount of said ingredients required for obtaining an active cosmetic product when reconstituted in a given amount of water. In particular embodiments, the solid form has a mass of between 5 and 25 grams and said given amount of water is between 10 and 250 ml. In particular embodiments, the cosmetic active agent is selected from at least one of a low molecular weight polysaccharide, panthenol, aloe vera extract, agave extract, allantoin, inulin, zinc salts, and copper salts. In particular embodiments, the low molecular weight polysaccharide is selected from chitosan or hyaluronic acid. In particular embodiments, the water-soluble preservative is selected from one or more of benzoic acid, levulinic acid, dehydroacetic acid, sorbic acid, and p-anisic acid. In particular embodiments, the pH modifying agent is a weak acid or a natural buffer.

In particular embodiments, the form of the composition in solid form is selected from a tablet or a sphere.

It is envisaged that the compositions described herein, when reconstituted, generate a fluid cosmetic product which can have different applications, such as a make-up remover, such as a micellar make-up melt-away, a face cleanser, such as an ultra-gentle soap-free cleanser, a face toner, such as a pH-balancing toner, a face scrub, such as a microdermabrasion facial scrub, a face care product, such as an anti-oxidant moisturising face care, an anti-pollution hydrating mist for face, hair and/or body mist, a deodorant, a hand and/or body soap, a solar skin protection product, such as a photo defence and bio protection product, an anti-mosquito product, yoga mat cleaning spray, face moisturizer, face mask or a face serum. In particular embodiments the solid composition form comprises a demarcation on at least a part of its surface indicating a name or brand. The demarcation can be two-dimensional, e.g. a printed sign or word, typically of a name or brand, provided on the surface. The demarcation can also be three-dimensional, i.e. in the form of an indentation or a ridge on the surface of the solid form.

A further aspect of the invention relates to a kit comprising two or more of the compositions in solid form as described herein and at least one empty vial suitable for holding and dispensing a fluid cosmetic. In particular embodiments, the kit further comprises at least one vial head suitable for dispensing the fluid cosmetic.

A further related aspect provides methods for producing a fluid cosmetic product, said method comprising dissolving a composition in solid form as described herein, comprising said cosmetic active in a concentration corresponding to the concentration required for said fluid cosmetic product in a given volume, in a volume of water corresponding to said given volume.

A further related aspect provides methods for producing the solid form cosmetic as described herein comprising the steps of (a) grinding the ingredients having particle size larger than 0.2 mm; (b) blending the ingredients to a homogenous mass; (c) providing the homogenous mass in a mold; pressing the mass in the mold; and (d) unmolding the solid form. In particular embodiments, step (d) comprises pressing the mass with a stamp press so as to obtain a logo or a text on the solid form. The invention thus also encompass solid form cosmetic obtained or obtainable by the methods of the present invention.

### BRIEF DESCRIPTION OF THE FIGURES

FIG. 1 examples of solid form compositions according to particular embodiments of the invention.

FIG. 2 shows/illustrates an embodiment of a method of making the solid form composition of the invention.

FIG. 3 shows/illustrates a method for reconstituting a fluid cosmetic according to an embodiment of the invention.

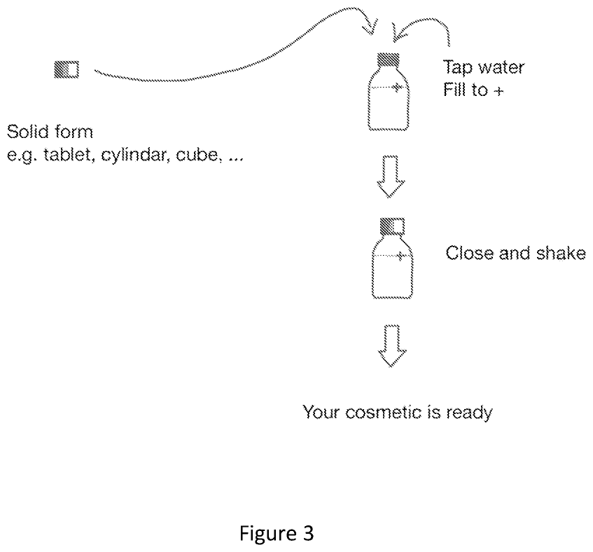

### DETAILED DESCRIPTION

The discussion of the background to the invention herein is included to explain the context of the invention. This is not to be taken as an admission that any of the material referred to was published, known, or part of the common general knowledge in any country as of the priority date of any of the claims.

Throughout this disclosure, various publications, patents and published patent specifications are referenced by an identifying citation. All documents cited in the present specification are hereby incorporated by reference in their entirety. In particular, the teachings or sections of such documents herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, definitions for the terms used in the description are included to better appreciate the teaching of the present invention. By means of further guidance, term definitions are included to better appreciate the teaching of the invention. When specific terms are defined in connection with a particular aspect of the invention or a particular embodiment of the invention, such connotation or meaning is meant to apply throughout this specification, i.e., also in the context of other aspects or embodiments of the invention, unless otherwise defined.

As used herein, the singular forms “a,” “an” and “the” include both singular and plural referents, unless the context clearly indicates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms also encompass “consisting of” and “consisting essentially of”, which enjoy well-established meanings in patent terminology.

The recitation of numerical ranges by endpoints includes all integer numbers and, where appropriate, fractions subsumed within that range (e.g. 1 to 10% can include 1%, 2%, 3%, 4%, 5%, 6%, 7%, 8%, 9% when referring to, for example, a number of elements, and can also include 1.5%, 2.75%, 3.80% and 7.65%, when referring to, for example, quantities). The recitation of end points also includes the end point values themselves (e.g. from 1% to 10% includes both 1% and 10%). Any numerical range recited herein is intended to include all sub-ranges subsumed therein. This applies to numerical ranges irrespective of whether they are introduced by the expression “from . . . to . . . ” or the expression “between . . . and . . . ” or another expression.

The term “about” as used herein when referring to a measurable value such as a parameter, an amount, a temporal duration, and the like, are meant to encompass variations of and from the specified value, such as variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more members or at least one member of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members. In another example, “one or more” or “at least one” may refer to 1, 2, 3, 4, 5, 6, 7 or more.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art.

The term “cosmetic active agent” or “cosmetic active ingredient” as used herein refers to a molecule which can be used in the care for the face and body, or to accentuate or change a person's appearance. Cosmetic agents thus include, but are not limited to molecules having a positive effect on the cells of the skin, body and/or hair such as at least one of anti-wrinkle, anti-aging, anti-free radical, cell-promoting, strengthening the barrier function of the skin, protecting the skin and/or hair from external stimuli, skin irritation alleviation, skin soothing, improving skin tone etc.

The term “surfactant” as used herein refers to a surface active agent which reduces surface tension. For instance, surfactants may be, without limitation, sodium lauryl sulfate and sodium cocoyl isethionate.

Although all methods, compositions and kits are the same or equivalent to those described herein may be used in practice or in testing of the present invention, the preferred methods, compositions and kits will now be described.

Provided herein is a novel means of formulating cosmetic products for skin, hair and/or body in a compact and solid format. The solid product contains one or more cosmetic actives in concentrated form, for dispersion in a specified amount of fluid prior to use. The formulation in a solid form reduces packaging size and waste.

The concept of the invention is that the composition is provided in compact form but that all of the components of the composition are soluble, such that when a fluid such as water is added to the composition, this results in a product with a homogenous texture. In particular embodiments, the resulting product, when dissolved in water is a clear, i.e. transparent composition. In particular embodiments, the total transmittance of the product after dissolution is between 80-99%, preferably at least 90%, even more preferably more than 95%. The compositions of the invention further allow easy reconstitution in fluid such as water, by manual shaking of the vial without requiring a mixing apparatus. In particular embodiment, the reconstitution in the fluid by manual shaking occurs within 1-5 minutes, preferably within 1 minute.

More particularly, the present invention provides for a composition in solid form comprising one or more cosmetic active agents and, one or more carriers or agglomerating agents, one or more preservatives and optionally one or more water-soluble pH-modifying agents and/or one or more water-soluble surfactants.

The nature of the cosmetic agent is not critical to the invention. However, in the context of the present invention the active is preferably a solid. Moreover, technical effect is particularly advantageous in the context of cosmetic agents that are typically provided as a fluid in a vial. Examples of such cosmetics include a make-up removers, face cleansers, face toners, a face scrubs, and other face care products, deodorants, hand and/or body soaps. Examples of corresponding cosmetic active agents include, but are not limited to
Cleansing, mattifying, Anti-wrinkle, anti-redness, anti-oxidant, collagen-stimulating agents, such as sodium lauryl sulfate, sodium C14-C16 sulfonate; ethyl ascorbic acid, cocoyl glucoside, decyl glucoside, cocamidopropyl betaine, sodium methyl cocoyl taurate emollients such as petrolatum, lanolin, mineral oil, cocoglycerides, caprylic/capric triglycerides, PEG-6-caprylic/capric glycerides, isoprenyl myristate, dimethicone, ceramides and fatty alcohols such as cetearyl alcohol;
exfoliating agents such as benzoyl peroxide, citric acid, salicylic acid, alpha hydroxy acid (AHA) or fruit acid;
humectants such as glycerin, propylene glycol, sodium hyaluronate, retinol and sulfur.

In addition, an active ingredient may be selected from:
sunscreen agent, which can be organic such as benzophenone-3-butyl, methoxydibenzoylmethane, ethylhexyl methoxycinnamate, or inorganic such as titanium dioxide or zinc oxide;
bacteria, such as Lactobacillus, Bificobacterium, Vitreoscilla optionally combined with one or more prebiotic sugars like xylitol
probiotics, and
plant or algae extracts.

In particular embodiments, the cosmetic active agent in the compositions described herein comprises at least one or more of a cosmetic such as but not limited to a low molecular weight polysaccharide, panthenol, aloe vera extract, agave extract, allantoin, inulin, zinc salts, copper salts. Preferably, the active is a natural compound. In further particular embodiments, the cosmetic active agent is a low molecular weight polysaccharide selected from chitosan or hyaluronic acid. In particular embodiments, the composition is a skin care composition and comprises inulin as an active ingredient to boost the beneficial bacterial flora on the skin.

In particular embodiments, the composition comprises more than one active ingredient. More particularly, in particular embodiments, the composition comprises different actives with the same or different activities. For instance, a composition for use as a skin care cosmetic can comprise one or more actives with purifying and repairing function in combination with one or more actives, such as a probiotic and a moisturizer and/or in combination with one or more actives with purifying, anti-acne and/or seboregulator function. The nature of the active agent will be selected based on the intended use of the cosmetic, for instance, the following active agents or combinations of active agents can be envisaged:
for moisturizing serum with fresh texture: inulin, beta glucan in the tablet.
water bio-active anti-wrinkle: hyaluronic acid, inulin, vitamin C derivative (such as ethyl-ascorbic acid).
gentle repairing serum: inulin, allantoin.
purifying water: inulin
cleaning water: tenside, inulin
hair care serum: inulin.

In particular embodiments, the cosmetic is a face-care product, and the one or more active ingredients are inulin, aloe vera and zinc.

The active ingredient is preferably present in a concentration of between 0.1-10%, such as between 1-10%, between 2-8% between. In some embodiments the active ingredient is present in the composition in a concentration of between 3-5%. In some embodiments the active ingredient is present in the composition in a concentration of between 0.1-3%. In particular embodiments several active ingredients are combined. In these embodiments the total amount of active ingredient may be between 0.1 and 25%. As detailed herein, in particular embodiments, for instance when the product is a cleaning product, one or more of the active ingredients may be combined with a surfactant, which ensures cleaning and foaming, such as for instance polyglyceryl-10 laureate. In further embodiments, when the product is not a cleaning product, the composition does not comprise a surfactant which ensures cleaning and foaming.

The compositions of the present invention further comprise one or more carriers. The carrier molecules make up the base of the composition. The carrier molecules can have one or more additional functions such as an agglomerating aid or agent, a structuring aid and/or a dispersion aid. Agglomerating aids or agents are molecules that can agglomerate particles together. The nature of the agglomerating agent is not critical to the invention but will determine the nature of the cosmetic upon reconstitution. Examples of suitable agglomerating agents include but are not limited to (modified) starches, maltodextrins. In particular embodiments, the agglomerating agent is maltodextrin. A structuring agent stabilizes the structure and increases temperature resistance in anhydrous formulations. In particular embodiments, the agglomerating agent is also a structuring aid. A dispersant or a dispersing aid or agent is a substance, typically a surfactant, that is added to a suspension of solid or liquid particles in a liquid (such as a colloid or emulsion) to improve the separation of the particles and to prevent their settling or clumping. Examples of dispersing agents include but are not limited to compositions comprising oligosaccharides such as disaccharides such as sucrose. In particular embodiments, the carrier which is the dispersing agent can be cane sugar. In particular embodiments the carrier comprises both a structuring/agglomeration agent and a dispersion agent. Indeed, the inventors have combined the agglomerating/structuring effect of the agglomerating agent with a dispersion agent so as to reach a good equilibrium between the tablet solidity and its capacity to disperse in water.

It will be understood that in the context of the invention it is of interest to include both agglomerating agents to keep the composition compact in dry form and dispersion agents which improve the dispersion capacity of the composition after it is contacted with liquid. Ideally these agents will also function as structuring agents. In particular embodiment a further structuring agent may be added.

The amount of carrier in the composition is envisaged to be between 40-90 weight %. In particular embodiments, the composition comprises between 20-90% preferably between 25-45% of a agglomerating agent and/or between 20-90%, preferably between 25-45% of a dispersion agent. In particular embodiments, the composition comprises between 25-45% structuring agent and between 25-45% dispersion agent. In particular embodiments, the composition comprises between 25-45% carrier which functions as both a structuring aid and a agglomerating aid and between 25-45% carrier which functions as a dispersion aid. In particular embodiments, the composition comprises between 25-45% polysaccharide such as maltodextrine and between 25-45% cane sugar.

The compositions of the present invention may further comprise a water-soluble pH-modifying agent. The nature of the water-soluble pH modifying agent is not critical to the invention, it can be either a base or an acid. In particular embodiments, the acid is citric acid or lactic acid. In particular embodiments, the base is potassium hydroxide, sodium hydroxide or triethanolamine.

In particular embodiments, the pH modifying agent is an acid, such as citric acid. In order to ensure the skin's barrier function, an acidic environment on the skin is recommended, particularly where a skin-care product for use on older skin is envisaged. Each decade skin becomes more alkaline, resulting in a variety of undesirable changes and skin disorders ranging from wrinkles to pigmentation and even acne. Keeping the pH level of the skin around 5.5, thus slightly acidic, is optimum. Accordingly, in particular embodiments the cosmetic, when reconstituted has a pH of about 5, +/−0.5, such as 5.2-5.5. In particular embodiments, this is achieved by the combination of active ingredients. In particular embodiments, this is achieved by the inclusion of pH modifying agents such as citric acid.

In particular embodiments the compositions described herein comprise at least one water-soluble pH modifying agent in an amount between 0.1 and 10% in relation to the total weight of the composition, preferably between 0.1 and 5%, more preferably 0.2, 0.3, 0.4, 0.5, 0.6, 0.7, 0.8, 0.9, or between 1 to 10%. The pH modifying agent in the compositions described herein is selected from weak acids or natural buffers. For instance, citric, lactic, salicylic and pyruvic acids are preferred to be used at low strength, such as between 0.1 and 10%, but in other compositions may for instance be up to 12 percent, or as high as 20 percent. In particular embodiments the compositions as described herein do not comprise a pH modifying agent.

The composition may also contains one or more surfactants, dispersants or solubilizers (generally referred to herein as “surfactant”). Surfactants may have different properties. When the product is a cleansing products the surfactant is typically one that ensures foaming. The nature of the surfactant is not critical to the invention, other than that it is water-soluble. In particular embodiments, the surfactants may comprise one or more of a fluorine-based surfactant, a silicone-based surfactant, or an anionic surfactant. In a preferred embodiment, the surfactant is a fluorine or silicone-based surfactant.

Surfactants are also known to be active ingredients in skin care products. For instance, surfactants may have anti-wrinkle, anti-redness, cleansing, foaming and other activities. It will be understood that in these embodiments, the composition may but need not contain further active ingredients in addition to one or more surfactants. Examples of active surfactants include but are not limited to sodium lauryl sulfate, disodium lauryl sulfosuccinate, sodium C14-C16 sulfonate; polyglyceryl-10 laurate, cocoyl glucoside, decyl glucoside, cocamidopropyl betaine, cocoamphocarboxyglycinate, alpha olefin sulfonate and sodium methyl cocoyl taurate. In particular embodiments, the compositions comprise at least one water-soluble surfactant, in an amount between 1 and 20% in relation to the total weight of the composition, preferably between 5 and 10%, more preferably 1, 2, 3, 4, 5, 6, 7, 8, 9, 10, 11, 12, 13, 14, 15, 16, 17, 18, 19 or 20%. Surfactants for use in cosmetics are widely described. In particular embodiments, the active surfactant is combined with another active agent. For instance, a cleaning and foaming surfactant is combined with an active agent which boosts the bacterial flora on the skin. For instance, in particular embodiments polyglyceryl-10 laurate is combined with inulin.

In particular embodiments however, the composition does not comprise a surfactant and/or a lubricant. In particular embodiments, the composition does not comprise a surfactant other than one with cleansing and/or foaming properties. More particularly, the composition typically will not comprise components which act as a surfactant or lubricant only. Thus, the composition may comprise e.g. an agglomerating agent which also acts as a lubricant, but typically will not comprise an agent which ensures slipping or lubrication only. Similarly, preferably the composition will not comprise a surfactant which merely ensures foaming. It is noted in this regard that lubricants moreover are typically not water-soluble and will compromise the transparency of the resulting product.

Preferably a surfactant for use in the described compositions is selected from anionic or cationic surfactants, preferably selected from an alkyl sulfate, an alkyl ether carboxylate, an isethionate, an alkyl sulfosuccinate, an alkyl ether sulfate, an alkyl sulfonate, and/or an amino acid based surfactant, more preferably an alkylsulfosuccinate and/or an amino acid based surfactant.

The composition may also comprise one or more emulsifiers. Typically emulsifiers are used to mix water with oils. Accordingly, the addition of emulsifiers is particularly preferred when the composition also comprises essential oils. In these embodiments, the amount of emulsifier added may be between 2-20 weight % of the total composition, such as around 15%. Preferred emulsifiers are organic emulsifiers such as sucro-esters as cetearyl glucoside, sucrose stearate, . . . etc, glycero-esters such as polyglyceryl-10 laurate, polyglyceryl-3 caprylate, . . . etc, and other emulsifiers such as but not limited to cetearyl alcohol, sorbitan olivate and cetearyl olivate,

The composition preferably also contains one or more preservatives. The nature of the preservative is not critical to the invention, other than that it is water-soluble. In particular embodiments, the one or more preservative comprises one or more of benzoic acid and salts of benzoic acid (such as sodium benzoate), proprionic acid and its salts, salycic acid and its salts, hexa-2, 4 dienoic acid and its salts, potassium sorbate, formaldehyde, paraformaldehyde (or any other preservative listed in the 1223/2009/EC directive) as well as non-listed preservatives such as anisic acid, levulnic acid, glycerol caprylate. In particular embodiments the composition comprises the preservatives is potassium sorbate and sodium benzoate.

In particular embodiments, the compositions described herein comprise at least one water-soluble preservative in an amount between 0.1 and 10% in relation to the total weight of the composition, preferably between 0.1 and 10%, such as between 1-5%, more preferably 1, 2, 3, 4, 5, 6, 7, 8, 9 or 10%. In particular embodiments, the water-soluble preservative in the compositions described herein is selected from one or more of potassium sorbate, sodium benzoate, benzoic acid, levulinic acid, dehydro-acetic acid, sorbic acid, p-anisic acid. In particular embodiments, the composition comprises two or more different preservatives, wherein the combined amount of preservatives corresponds to the amount of the preservatives detailed above.

In particular embodiments, one of the other components recited above, may also function as a preservative. For instance, in particular embodiments, the emulsifier may also function as a preservative.

It will be understood that in the context of the present invention, while the ingredients are defined functionally, a given ingredient may have more than one function. For instance, in particular embodiments, the active ingredient may also have a pH modifying or emulsifying effect. Similarly, an emulsifier may also have a preservative effect.

In particular embodiments, the composition in solid form comprises:
40-90% of an carrier, such as 20-90%, preferably 25-45% an agglomerating agent, preferably maltodextrine and 20-90%, preferably 25-45% of a dispersion aid, such as cane sugar,
1-10% water-soluble preservative,
1-10% of one or more water-soluble and dispersible cosmetic active agents; and
optionally 0.1-10% of one or more water-soluble pH-modifying agents,
optionally 1-20% of one or more water-soluble surfactant.

According to particular embodiments, the compositions described herein comprises a carrier agent in an amount between 40 and 90% in relation to the total weight of the composition, preferably between 40 and 70%, more preferably 40, 45, 50, 55, 60, 65, 70, 75, 80, 85, or 90%. In particular embodiments, the carrier includes both an agglomerating agent and a dispersing agent, whereby each are present in about 20, 25, 30, 35, 40, or 45%. In particular embodiments the compositions consist of the ingredients recited above.

In particular embodiments, the composition in solid form comprises:
40-90% of an agglomerating agent, preferably maltodextrine,
1-10% water-soluble preservative,
0.1-10% of one or more water-soluble pH-modifying agents,
1-20% of one or more water-soluble surfactant and
1-10% of one or more water-soluble and dispersible cosmetic active agents.

According to particular embodiments, the compositions described herein comprises an agglomerating agent in an amount between 40 and 90% in relation to the total weight of the composition, preferably between 40 and 70%, more preferably 40, 45, 50, 55, 60, 65, 70, 75, 80, 85, or 90%.

The solid form compositions of the present invention are envisaged for reconstitution in a given amount of liquid, so as to obtain a fluid cosmetic. The amount of the one or more cosmetic active agents present in the composition is determined by the amount of cosmetic active agent envisaged in the reconstituted cosmetic. Similarly, the amount of said water-soluble preservative, and optionally said water-soluble pH modifying agent and/or water-soluble surfactant, in said solid form corresponds to the amount of said ingredients required for obtaining an active cosmetic product when reconstituted in a given amount of water. The solid forms as such are not suitable as cosmetics, given that they are solid and that the amount of cosmetic active agent is not suitable for direct use.

In particular embodiments the ingredients listed above make up 100% of the composition. However, in particular embodiments, one or more additional ingredients in addition to the ingredients listed above, such as one or more of a (natural) fragrance, a coloring agent, an antibacterial agent, an antifungal agent, a vitamin.

The compositions of the present invention may for instance comprise one or more additional ingredients such as one or more fragrances. Different examples of suitable fragrances are known in the art and include essential oils, such as but not limited to essential oils made from natural products such as from Citronella, lime, Lemon Grass, bergamot, Palmorsa, Ventiver, Patchouli, Jasmine, Marigold, Lilly of the Valley, Sugandhmantri, Néroli etc. . . . In particular embodiments, the composition comprises between 0.01-5% essential oils, preferably between 0.1-5% essential oils, such as 0.3%. In particular embodiments, the composition comprises 0.3% Néroli essential oil.

In particular embodiments, the compositions of the present invention further contain a color pigment or a glittering agent. In particular embodiments, the composition comprises between 0.01-5% of a color pigment.

In particular embodiments most or all of the ingredients of the composition are natural ingredients.

In particular embodiments, the solid form composition of the invention does not comprise an effervescent agent.

The compositions described herein are envisaged for external use only and as such are in most embodiments non-edible. The composition is in the form of a compact solid and does not contain water or other aqueous solutions.

In particular embodiments, said solid form has a given mass and is envisaged for reconstitution in a given amount of water. In particular embodiments, the solid form composition has a mass of between 5 and 25 grams and is envisaged for reconstitution in an amount of liquid between 5 and 1000 ml, preferably between 10 and 500 ml, more preferably between 10 and 250 ml or between 25 and 100 ml, or between 50 and 150 ml. According to specific embodiments, the compositions in the solid form are dosed to be used as either 1 unit per 80-125 ml liquid, such as one unit per about 100 ml water. Alternatively, if a vial of 200 ml is used, 2 units may be required if the unit dose is 1 unit per 100 ml.

According to particular embodiments, the compositions described herein comprise at least one water-soluble cosmetic active agent in an amount between 1 and 10% in relation to the total weight of the composition, preferably between 3 and 6%, more preferably 1, 2, 3, 4, 5, 6, 7, 8, 9 or 10%. Upon dissolution of the composition in solid form in the specified volume of the liquid, a cosmetic is obtained with suitable properties for use on the skin.

The solid form compositions of the invention are envisaged for reconstitution in water so as to obtain a cosmetic. In particular embodiments said cosmetic is a skincare product, a haircare product or a bodycare product, whereby the ingredients of the solid form composition as described above are selected so as to ensure reconstitution in said skincare product, a haircare product or a bodycare product. In further particular embodiments, the cosmetic is selected from
(a) a make-up remover, such as a micellar make-up melt-away,
(b) a face cleanser, such as an ultra-gentle soap-free cleanser,
(c) a face toner, such as a pH-balancing toner,
(d) a face scrub, such as a microdermabrasion facial scrub,
(e) a face care product, such as an anti-oxidant moisturizing face care,
(f) an anti-pollution hydrating mist for face, hair and/or body mist,
(g) a deodorant,
(h) a hand and/or body soap,
(i) a solar skin protection product, such as a photo defense and bio protection product,
(j) an antimosquito product,
(k) a yoga mat cleaning spray,
(l) a face moisturizer, or face mask
(m) a face serum, such as an intensive bio elixir with gorgeous plumping actives.

The nature of the solid form of the solid form composition of the invention is not critical. Envisaged forms of the solid-form composition include but are not limited to a tablet, a sphere, a cube, a lozenge etc.

In particular embodiments the solid form is a sphere with a diameter of between 1 and 7 cm, preferably between 2 and 3 cm. In particular embodiments, the diameter is at least 1.5 cm.

The tablet as described herein have particular dimensions, for instance when the tablet is a in the form of a cube, the dimensions are for instance about 2 cm×2 cm, but essentially any size is envisaged.

In particular embodiments, the solid form is in the shape of a tablet, with a height of at least 2 mm and a diameter of at least 5 mm. In a particular embodiment, the solid form is in the form of a cylinder with dimensions of about 1.0 to 3.0 cm in diameter, a height between 1.0 and 4.0 in height and a volume ranging from between 0.9 to 28.3 cm³. In a most preferred embodiment a cylindric tablet is envisaged having a diameter of about 2.0 cm, a height between 2.25 and 3.06 cm and a volume ranging from between 7.07 to 9.62 cm³.

The solid-form composition of the present invention further has a number of characteristics which are of interest in the context of the present invention. Particularly, the properties of the solid form composition are such that they promote dispersion or emulsion of the composition in the fluid, when contacted with said fluid.

Further features of the solid-form compositions of the present invention include compactness, not irritating, not greasy and firm at room temperature (not melting) (easily handled) homogeneity, solidity, solubility in fluid, such as water, etc.

A further aspect of the present invention relates to methods for producing a cosmetic product, using the solid form compositions of the present invention.

The invention thus also relates to a method for producing a cosmetic product, said method comprising dispersing a composition in solid form as described herein in a fluid. More particularly, the composition comprises a cosmetic active in a concentration corresponding to the concentration required for said cosmetic active in the final cosmetic product. Accordingly, when the solid form composition is dispersed in a given volume, said cosmetic is reconstituted. Dispersing the solid form composition preferably occurs in a vial, more particularly a vial which is suitable for holding and dispensing the cosmetic product after reconstitution. In particular embodiments, the predetermined amount of fluid is introduced into the vial first, after which the solid form composition is added to the vial. Alternatively, the solid form composition is introduced into the vial first, after which the fluid is added. In particular embodiments, the method may comprise shaking the vial so as to promote dispersing or emulsion of the solid form composition. In further embodiments, the method may involve shaking of the vial. In particular embodiments, the method may comprise stirring with a stirring tool in the vial to further dissolution of the solid form composition. In particular embodiments, the method may comprise grinding the solid-form composition with a grinding tool. In particular embodiments, the method comprises one or more of the steps described above.

In particular embodiments, the pressing does not require special techniques, special molds, intense forces, or temperature control. Indeed the preparation of the solid form compositions requires only pressure (that can be mechanical with a lever or an actuator) and simple molds (i.e. steel undercutting is enough, the shapes typically do not require complicated design and the number of pieces of the mold are reduced to three: punch, matrix, bottom. This is in contrast to complicated molds, which require the opening of several pieces to free the tablet, or which require steel guides to bring the punch to the right place. This is an important cost-advantage and logistics advantage of the production methods of the present invention.

In particular embodiments, the fluid is a water-based composition such as tap water, bottled water, distilled water, de-ionized water, sparkling water.

In particular embodiments, the fluid is a buffered solution.

As detailed above, the solid-form compositions of the present invention are envisaged for reconstitution in a given volume of liquid. A further aspect of the present invention relates to methods for producing a fluid cosmetic which comprise dissolving a composition in solid form as described herein comprising one or more cosmetic actives in a concentration corresponding to the concentration required for said cosmetic product in a given volume, whereby the composition in sold form is dissolved in a volume of water corresponding to said given volume. The methods of the invention may comprise providing an empty vial, filling the empty vial with the given volume of a fluid, such as tap water, adding the solid form composition and dissolving the solid form composition in the given volume of fluid in the vial. In particular embodiments, the method comprises closing the vial with a cap and shaking the vial to further dissolution of the solid form composition. In particular embodiments, the vial which contains a demarcation indicating the level of the fluid, for the given volume of fluid when provided in the vial. The compositions are characterized in that the resulting products can be reconstituted by simply dissolving the composition in solid form in a fluid requiring only simple manual mixing.

As detailed above, the solid-form compositions of the present invention are envisaged for reconstitution in a given volume of liquid so as to obtain a fluid cosmetic. In order to ensure reconstitution in the right amount of fluid, appropriate vials can be provided which (a) allow easy reconstitution of the solid form compositions of the invention and optionally (b) allow easy proportioning of the reconstituted cosmetic.

Accordingly, a further aspect of the invention provides a kit, comprising the compositions of the present invention and a vial, more particularly a vial suitable for reconstitution of the cosmetic, i.e. which ensures that the composition can be reconstituted in the appropriate volume of fluid. In particular embodiments, the vial is an empty vial which can be filled with the appropriate amount of the suitable liquid. More than one solid-form compositions can be reconstituted consecutively using the same vial, allowing a reduction in packaging. Hance, in a particular embodiment, the kit of the present invention comprises two or more of the compositions in solid form according to the invention and at least one empty vial or container suitable for holding a fluid cosmetic.

Preferably, at least one vial is provided together in the kit together with one or more of the compositions in solid form. More preferably 6, 12 or 24 compositions in solid form are provided in one kit. In particular embodiments, the vial is an empty vial.

The vial provided in the kit described herein is re-fillable. The vial may be proportioned to contain a volume between 50 and 1000 ml, preferably between 100 and 500 ml, or between 150 and 250 ml. The vial may contain any volume as necessary for reconstituting and holding a fluid cosmetic. The vial may be in glass, in ceramic, in stainless steel, made of plant based-plastic, or may be a mix of 2 or 3 materials.

In particular embodiments, the vial allows for easy dispensing of the fluid cosmetic. The shape of the vial is not critical. However, in particular embodiments, the opening of the vial is such that it allows for easy passage of the solid form composition into the vial. In further particular embodiments, the vial allows portioned dispensing of the fluid therein. Typically, this implies the provision of a dispenser cap or vial head, such as pump-dispenser, a spray etc. Thus in particular embodiments, the kit of the present invention further comprises a vial head, more particularly a pump, a roll-on or a spray head. The kits described herein may thus comprise at least one vial head, which is adapted to be used in combination with the fluid cosmetic.

In particular embodiments, the vial comprises a demarcation as to the appropriate level of fluid that is to be used for reconstitution of the fluid cosmetic. In further embodiments, the vial comprises multiple demarcations corresponding to the reconstitution of one or more volumes of the fluid cosmetic to be reconstituted. Typically the demarcation is a visual element on the side of the vial, such as a horizontal line or cross.

In particular embodiments, the kit comprises more than one vial head, such that an appropriate vial head can be selected.

In particular embodiments, the kit may comprise a device for grinding the solid-form composition of the invention and/or for stirring in the vial, so as to promote dispersion or emulsion of the composition in the vial. In particular embodiments, the device is a combined tool for grinding and stirring. In particular embodiments, the device has a length which allows it to be introduced towards the bottom of the vial, i.e. its length is longer than that of the vial present in the kit.

In particular embodiments, the kit comprises more than one empty vial and more than one composition in solid form as described herein. The more than one composition in solid form can be the same or can be different. For instance, two different compositions in solid form, each for a distinct cosmetic application, are present in the kit. For instance the kit may comprise at least two, preferably at least three, at least four or at least five distinct compositions in solid form selected from compositions which can be reconstituted to a make-up remover, a face cleanser, a face toner, a face scrub, a face care product, an anti-pollution hydrating mist for face, hair and/or body, a deodorant, a hand and body soap, a solar skin protection product, an anti-mosquito product, and a face serum.

In particular embodiments, the kit comprises an additional container comprising a liquid suitable for reconstitution of the solid-form composition of the invention into a cosmetic as described herein. Preferably, the container is of a size such that it contains sufficient fluid for the reconstitution of more than one solid composition of the present invention.

In particular embodiments of the kits according to the present invention, a leaflet is provided, which describes how the solid-form composition of the invention can be reconstituted to a cosmetic product using the vial and a fluid as described herein, using the method for the production/reconstitution of a cosmetic as described herein. Preferably, the leaflet describes one or more of the solid-form composition, the vial, the vial head and the container present in the kit.

A further aspect of the invention relates to methods of producing the solid form composition of the present invention described herein. More particularly, the invention provides methods for producing solid form compositions suitable for reconstitution to a cosmetic.

In particular embodiments said method for producing the solid form cosmetic product comprises grinding, blending, pressing and unmolding steps. In further particular embodiments, the methods comprise the steps of:
(a) grinding the ingredients of the solid form composition as described herein having particle size larger than 0.2 mm,
(b) blending the ingredients to a homogenous mass,
(c) providing the homogenous mass in a mold,
(d) pressing the mass in the mold; and
(e) unmolding the solid form.

Depending on the nature of the composition, at least some of the ingredients are particulate (provided as powder or granules), while others are provided as liquid/oil. Preferably, most of the ingredients are provided in solid form, i.e. as particulate. According to particular embodiments of the invention, the grinding of the particular ingredients is done using a grinder. The grinder may be any grinder known in the art, such as for example a MultiDrive basic grinder from IKA, or a larger grinder for industrial use.

In particular embodiments, the methods of the invention further comprise mixing of the oily ingredients separately, whereafter the blended oily mixture is blended with the ground ingredients to a homogenous paste.

The molds used in the context of the present invention ensure the shape of the solid-form compositions of the present invention. The mold die can be any mold known in the art, suitable for molding a homogenous mass made up of the ingredients described herein, such as for instance a Chigods molding structure.

The step of pressing the mass in the mold is typically ensured using a press. In particular embodiments, the mass is pressed in the mold using a pressure of about 100-1000 kg.

In particular embodiments, the method step (d) described above comprises pressing the mass with a stamp press so as to obtain a logo, a brand or a text on the solid form. Alternatively the logo, brand or text can be printed on the solid form in a separate step after step (d) or (e) of the method described above.

In particular embodiments, the method may comprise a quality control step which involves checking one or more of the shape, density, homogeneity, solidity, solubilisation time of the solid form composition. In particular embodiment, the method comprises determining that the composition is capable of being completely dissolved in water at room temperature in a time period between 0-30 min.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as follows in the spirit and scope of the appended claims.

The herein disclosed aspects and embodiments of the invention are further supported by the following non-limiting examples.

### EXAMPLES

### Example 1: Formulation of a Skin Care Product into a Solid-Form Composition

For an active cosmetic which is a cleaning product which hydrates the skin and protects useful commensal bacteria, the following ingredients were combined (percentages are rounded off):
carrier/agglomerating agent: maltodextine: 24%
carrier/dispersing agent: cane sugar: 39%
active with hydration effect and protective effect for useful commensal bacteria: inulin: 16%
pH modifying agent: citric acid: 1%
essential oil: 0.3%
emulsifier: polyglyceryl-3 caprylate: 8%
emulsifier: polyglyceryl-10 laureate: 6%
preservative: potassium sorbate: 2.5%
preservative: sodium benzoate: 2.5%

The solid ingredients are ground in a grinder; the liquid or pasty ingredients (essential oils, emulsifiers) are mixed together. The ground ingredients are blended with the remainder of the ingredients to a homogenous mass. The homogenous mass is then provided in a tablet mold, and the mass is then pressed in the mold.

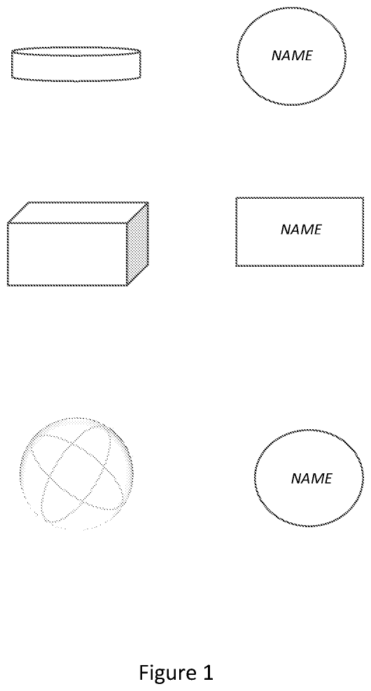

Finally, the solid form is unmolded so as to obtain the solid form of 10.5 g. Examples of solid forms are provided in FIG. 1.

Solid form compositions are made for skin care products for different types of skin, wherein the ingredients were adjusted thereto. The presence of the surfactant which has cleaning and foaming activities ensures that the resulting product will have cleansing and foaming properties.

### Example 2: Formulation of a Skin Care Product into a Solid-Form Composition

For an active cosmetic for treatment of the skin, the following ingredients were combined:
carrier/agglomerating agent: maltodextine: about 40%
carrier/dispersing agent: cane sugar: about 40%
actives: ethyl ascorbic acid: about 1%+chicory root extract: about 1% (anti-wrinkle, anti-oxidant, collagen stimulating)
pH modifying agent: about citric acid: about 1%
tangerine and lichee essential oils: about 2.5%
emulsifier: polyglyceryl-3 caprylate: about 8%
preservative: potassium sorbate: 2.5%
preservative: sodium benzoate: 2.5%

The solid ingredients are ground in a grinder; the liquid or pasty ingredients (essential oils, emulsifiers) are mixed together. The ground ingredients are blended with the remainder of the ingredients to a homogenous mass. The homogenous mass is then provided in a tablet mold, and the mass is then pressed in the mold.

Finally, the solid form is unmolded so as to obtain the solid form of 10.5 g. Examples of solid forms are provided in FIG. 1.

### Example 3: Formulation of a Skin Care Product into a Solid-Form Composition

For an active, regulating cosmetic for greasy skin, the following ingredients were combined
agglomerating agent: maltodextrine 4.9 g
active with purifying and repairing function: aloe vera 0.5 g
active, probiotic+moisturizer: inulin 1 g
active, purifying+anti-acne+seboregulator: zinc 0.1 g
preservatives: sodium anisate 0.3 g and polyglyceryl-3 caprylate 1 g
dispersing agent/solubilizer of essential oils: capryl/caprylic glucoside+sodium cocoyl glutamate+other tensides: 2 g
essential oil for the perfume: may chang 0.1 g
essential oil for the perfume: corn mith 0.5 g
pH adjustor: citric acid 0.1 g

The solid ingredients are ground in a grinder; the liquid or pasty ingredients (essential oils, capryl/caprylic glucoside+sodium cocoyl glutamate+other tensides) are mixed together. The ground ingredients are blended with the remainder of the ingredients to a homogenous mass. The homogenous mass is then provided in a tablet mold, and the mass is then pressed in the mold.

Finally, the solid form is unmolded so as to obtain the solid form of 10.5 g. Examples of solid forms are provided in FIG. 1.

Solid form compositions are made for skin care products for different types of skin, wherein the ingredients were adjusted thereto.

### Example 4: Making of a Cosmetic Using the Composition in Solid Form According to the Invention

The compositions of the invention produced as described in example 1, 2 or 3 are used for the production of a cosmetic. More particularly, one solid form composition is provided in a suitable vial comprising a liquid. More particularly, an empty 100 ml vial is filled with 100 ml deionized water, after which the solid form composition of example 1 is introduced therein. The vial is closed with a cap or vial head and shaken to promote dispersion or emulsion of the solid form composition.

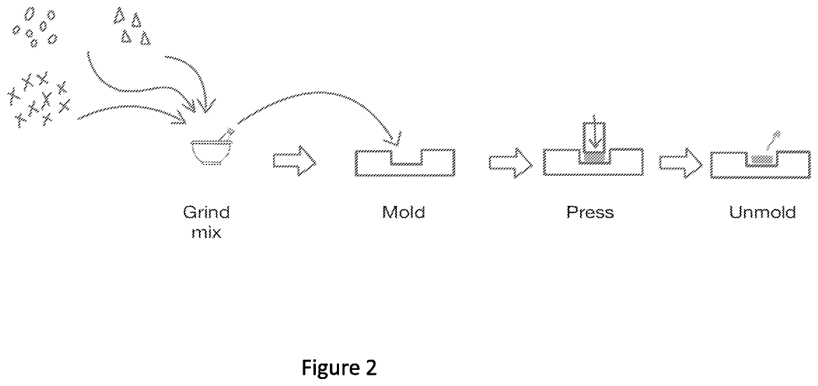

An exemplary method is described in FIG. 2.

1. A composition in solid form for dissolving into a fluid cosmetic comprising
40-90% carrier, preferably comprising 20-45% agglomerating agent and 20-45% dispersing agent
1-10% water-soluble preservative,
0.1-10% of one or more water-soluble pH-modifying agents; and
1-20% of one or more water-soluble and dispersible cosmetic active agent

2. The composition according to claim 1 wherein said cosmetic active agent is an active agent for skincare.

3. The composition according to claim 1, wherein the amount of said water-soluble preservative, water-soluble pH modifying agent, and said water-soluble and dispersible cosmetic actives in said solid form corresponds to the amount of said ingredients required for obtaining an active fluid cosmetic product when reconstituted in a given amount of water.

4. The composition according to claim 3, wherein said solid form has a mass of between 5 and 25 grams and said given amount of water is between 10 and 250 ml.

5. The composition according to claim 1, wherein said cosmetic active agent is selected from at least one of a low molecular weight polysaccharide, panthenol, aloe vera extract, agave extract, allantoin, inulin, zinc salts, and copper salts.

6. The composition according to claim 1, wherein said agglomerating agent is maltodextrine and said dispersing agent is an oligosaccharide.

7. The composition according to claim 1, wherein the water-soluble preservative is selected from one or more of potassium sorbate, sodium benzoate, benzoic acid, levulinic acid, dehydroacetic acid, sorbic acid, and p-anisic acid.

8. The composition according to claim 1, wherein the pH modifying agent is selected a weak acid or a natural buffers.

9. The composition according to claim 1, further comprising essential oils and an emulsifying agent.

10. The composition according to claim 1, wherein said solid form is a tablet or a sphere.

11. The composition according to claim 1, wherein the reconstitution into water results into one of the following fluid cosmetics:
a. a make-up remover, such as a micellar make-up melt-away,
b. a face cleanser, such as an ultra-gentle soap-free cleanser,
c. a face toner, such as a pH-balancing toner,
d. a face scrub, such as a microdermabrasion facial scrub,
e. a face care product, such as an anti-oxidant moisturising face care,
f. an anti-pollution hydrating mist for face, hair and/or body mist,
g. a deodorant,
h. a hand and/or body soap,
i. a solar skin protection product, such as a photo defence and bio protection product,
j. an anti-mosquito product,
k. a yoga mat cleaning spray,
l. a face moisturizer or face mask, or
m. a face serum, such as an intensive bio elixir with gorgeous plumping actives,

12. A kit comprising two or more of the compositions in solid form of claim 1 and at least one empty vial suitable for holding and dispensing a fluid cosmetic.

13. The kit according to claim 12, which further comprises a vial head suitable for dispensing the cosmetic.

14. A method for producing a fluid cosmetic product, said method comprising dissolving a composition in solid form according to claim 1, comprising said cosmetic active in a concentration corresponding to the concentration required for said cosmetic product in a given volume, in a volume of water corresponding to said given volume.

15. A method for producing the solid form cosmetic according to claim 1 comprising
(a) Grinding the ingredients having particle size larger than 0.2 mm;
(b) Blending the ingredients to a homogenous mass;
(c) Providing the homogenous mass in a mold;
(d) Pressing the mass in the mold; and
(e) Unmolding the solid form.

16. The method of claim 15, wherein step (d) comprises pressing the mass with a stamp press so as to obtain a logo or a text on the solid form.
